(19) United States
(12) Patent Application Publication
(10) Pub. No.: US 2006/0058655 A1
(43) Pub. Date: Mar. 16, 2006

(54) Ultrasonic transducer having a thin wire interface

(75) Inventor: Blake W. Little, Bothell, WA (US)

Correspondence Address: DALLAS OFFICE OF FULBRIGHT & JAWORSKI L.L.P., 2200 ROSS AVENUE, SUITE 2800, DALLAS, TX, 75201-2784, US

(73) Assignee: SonoSite, Inc., Bothell, WA (US)

(21) Appl. No.: 10/925,114

(22) Filed: Aug. 24, 2004

(51) Int. Cl. A61B 8/00

(52) U.S. Cl. 600/437

(57) ABSTRACT

The present invention is directed to an ultrasound system and method which, in one embodiment, partitions the main body processing such that a portion of the processing is contained within the transducer thereby reducing the need for a multiplicity of high performance cables running between the transducer and the main body. This is possible through the use of a unique architecture to allow for proper power management given the small transducer size and an architecture that exploits the high levels of integration possible on integrated circuit technologies allowing for its implementation in a few highly integrated circuits with virtually no external components outside of the ICs.

### CROSS-REFERENCE TO RELATED APPLICATIONS AND PATENTS

The present application is related to co-pending, and commonly-assigned U.S. patent application Attorney Docket No. 65744/P017US/10404216, entitled “Ultra System Power Management,” filed concurrently herewith U.S. patent application Ser. No. 10/847,643, filed on May 17, 2004, entitled “Processing Of Medical Signals;” U.S. patent application Ser. No. 10/821,123, filed on Apr. 8, 2004, entitled “Systems And Methods For Providing ASICS For Use In Multiple Applications;” U.S. patent application Ser. No. 10/821,198, filed on Apr. 8, 2004, entitled “System And Method For Enhancing Gray Scale Output On A Color Display;” the disclosures of which are all hereby incorporated.

### TECHNICAL FIELD

This disclosure relates to ultrasound devices and more particularly to such devices having a thin wire interface.

### BACKGROUND OF THE INVENTION

Ultrasound medical devices are becoming more common. Their typical implementation has the transducer portion separate from the main processing unit of the device. Traditionally, the analog and digital signal processing of the raw ultrasound signals to/from a patient are performed in a main processing unit. The raw ultrasound signals are passed to/from the scanhead transducer across a cable to the main processing unit. The cable that connects the ultrasound transducer with the main body of the ultrasound processing unit must be fairly long because the processing unit is not easily moveable and the scanhead must be placed on the anatomy of interest in a variety of positions. The cable is also typically large and heavy because it carries the transmit and receive signals for a number of individual elements of the transducers, located in the transducer head. The length usually in excess of six feet, coupled with the weight of the cable places significant stress and strain on a sonographer. The cable also adds significant cost and complexity to the system. A typical ultrasound device is shown in U.S. Pat. No. 5,772,412 dated Mar. 3, 1998 and U.S. Pat. No. 6,471,651 dated Oct. 29, 2002 which patent is hereby incorporated by reference herein.

Another problem with existing cables is that they typically contain a large number of individual coaxial cables that are expensive and difficult to connect to a single connector. A connector is typically required on the cable since multiple tranducers are used on the system for different applications. The connector, due to the large number of interconnect lines and the sensitive nature of the signals, is therefore large, complicated and expensive. Thus, the overall cable is expensive, troublesome to assemble and repair as well as difficult to use.

The reason for multiple cables is that the individual elements of the transducers are individually excited with electronic wave forms so as to generate mechanical movement of the transducer elements thereby creating ultrasound energy which is then transmitted to the patient's body. The reflected energy from internal organs (and other items of interest) comes back to the transducer elements and is converted back to electrical signals for subsequent processing by the processing unit. The signal between the transducer and the processor unit must pass without significant distortion, attenuation or interference up and down the connecting cable.

### BRIEF SUMMARY OF THE INVENTION

The present invention is directed to an ultrasound system and method which, in one embodiment, partitions the main body processing such that a portion of the processing is contained within the transducer thereby reducing the need for a multiplicity of high performance cables running between the transducer and the main body. This is possible through the use of a unique architecture to allow for proper power management given the small transducer size and an architecture that exploits the high levels of integration possible on integrated circuit technologies allowing for its implementation in a few highly integrated circuits with virtually no external components outside of the ICs.

In one embodiment, the transducer processing consists of transmitters, receivers, and the beam formers necessary to control and generate the beam formed ultrasound signal. By partitioning the system in this manner the output of the scanhead now becomes a digital data stream. All the sensitive analog signals are maintained in close proximity to their transmitters receivers and transducer elements thereby eliminating any significant signal degradation allowing increased performance. The digital data stream can also be converted to a serial high-speed bit stream to further reduce signal count across the interface. The result is a cable and connector having an extremely low signal count. Also, the signals on the cable are digital and, therefore, the cable does not require as high a fidelity, thereby further reducing the cost and size of the cable and connector.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated that the conception and specific embodiment disclosed may be readily utilized as a basis for modifying or designing other structures for carrying out the same purposes of the present invention. It should also be realized that such equivalent constructions do not depart from the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 shows one embodiment of a prior art ultrasound system;

FIG. 2 shows one embodiment of an ultrasound system partitioned to allow for digital signaling between the transducer and the main processor; and

FIG. 3 shows one embodiment for further reducing the data bandwidth between the transducer and the main processor.

### DETAILED DESCRIPTION OF THE INVENTION

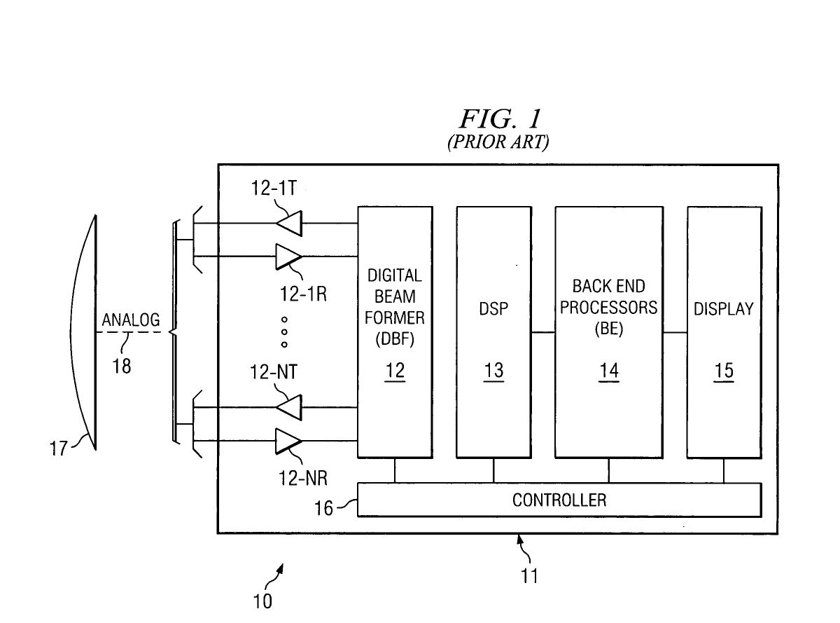

FIG. 1 shows a typical prior art architecture of an ultrasound system, such as system 10 having transducer array 17, which is coupled via analog cable 18 to individual receiving and transmit channels 12-IT, 12-IR to 12-NT, 12-NR to digital beam former 12. Typically, the Tx and Rx signals are time multiplexed. DSP 13 provides signals to and receives signals from beam former 12. Back end processing 14 then provides signals to drive display 15 all under control of controller 16. The operation of these elements can be as discussed in the above-identified '412 and '651 patents.

In this arrangement, cable 18 contains a high number of individual signals, typically carried on coax cables, usually in the order of 128 or 256 to carry the analog signals from transducer array 17 back and forth between receiving and transmit channels 12-IT, 12-IR to 12-NT, 12-NR. As discussed above, cable 18 is big, bulky, heavy, expensive and not very efficient. The analog signals are also sensitive, often requiring tuning to try to compensate for the loading of the cable.

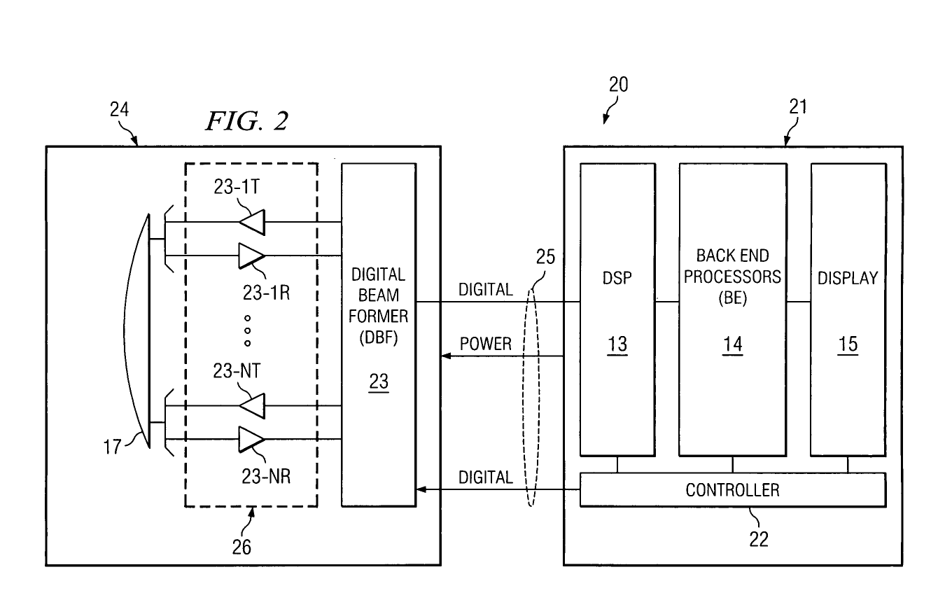

FIG. 2 shows one embodiment of ultrasound system 20 in which the interface between the beam former, such as beam former 23, and DSP 13 is moved to transducer 24. Beam former 23 drives transducer 17 via amplifiers and receivers, such as amplifiers 23-IT, 23-IR to 23-NT, 23-RT to/from beam former 23. This arrangement eliminates analog cable 18 (FIG. 1) replacing it with digital cable 25 which can be a much smaller cable since only a small number of wires are needed to provide necessary control. Digital cable 25 runs between processing unit 21 and transducer 24. Thus, elements 23 and 26 are within a common housing 24 with transducer 17.

In addition to cable size reduction, this rearrangement of elements also results in a performance gain. By eliminating cable 18 analog loading, distortion and attenuation characteristics are also eliminated allowing for increased performance and signal integrity. Better sensitivity, better response, and better bandwidth are achieved. In addition, this arrangement reduces power loss of the transmitters on the cable.

Cable 25 (or 33) is preferably a pair of Low Voltage Differential Signal (LVDS) lines to transmit the digital data back and forth. Also, a USB or USB2, or IEEE1394 type interface could also be used using USB on other now standard interface could be used. This interface could also be replaced with a wireless interface, if desired. However, for wireless given the present transmission bandwidths available it would be better to move additional DSP functions to the transducer as well, thereby even further reducing the data bandwidth required.

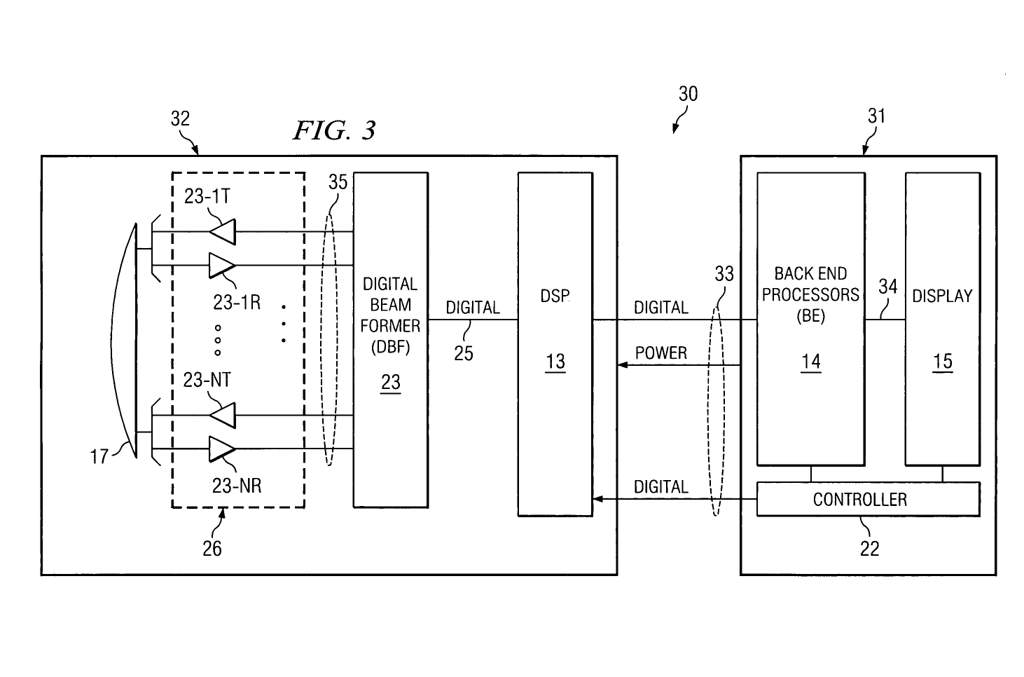

As shown in FIG. 3, the system can be partitioned into five processing blocks; transmit/receive (Tx/Rx) 26, digital beam former (DBF) 23, digital signal processor (DSP) 13, backend processing (BE) 14 and display 15. Pulser circuits, multiplexor circuits, low noise time gain control amplifiers and filters are integrated into Tx/Rx 26. Multiple A/D converters, digital beam forming circuits and control logic are integrated in DBF 23. One embodiment for accomplishing such an arrangement is shown in the above-identified application entitled, “Systems And Methods For Providing ASICS For Use In Multiple Applications.” DSP 13 consists of circuits required for echo and flow signal processing and includes analytic signal detection and compression, multi-rate filtering, and moving target detection capabilities. FIG. 3 also shows display 15 for display of data including image data. This display could be in the same housing as processor 14, or could be separate from both the processor and from the transducer.

In a preferred embodiment, DBF 23, DSP 13 and BE 14 would be implemented using digital CMOS ASICS and digital/analog mixed-mode ASICS and Tx/Rx 26 would be implemented based on high-voltage and/or Bi-Cmos technology. The total weight of the scanhead module of one embodiment is less than 12 ounces. Excluding the housing, transducer 17, in one embodiment, weighs less than 8 ounces. The peak power consumption is approximately 6 watts. Average power consumption with power management is less than 4 watts and the bandwidth of the signals over the interface from the transducer to the processing unit, has been reduced at least on order of magnitude from approximately 400 Mbps to under 40 Mbps. In one embodiment, for a video display having 128×512 pixels, a data rate of 16 Mbps is possible using the concepts discussed herein.

Although the present invention and its advantages have been described in detail, it should be understood that various changes, substitutions and alterations can be made herein without departing from the invention as defined by the appended claims. Moreover, the scope of the present application is not intended to be limited to the particular embodiments of the process, machine, manufacture, composition of matter, means, methods and steps described in the specification. As one will readily appreciate from the disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

What is claimed is:

1. An ultrasound system comprising a transducer for sending ultrasound energy into a body and for receiving from said body energy reflections of sent ones of said signals:
a processor separated form said transducer, said processor operable for processing said signals into a video display according to certain protocols; and
wherein said transducer comprises:
at least one scanhead;
a digital beam former;
a plurality of amplifiers for allowing signal energy to pass between said scanhead and said digital beam former; and
a digital cable for connection to said processor, said digital cable operable for communicating digitally between said beam former and said processor.

2. The ultrasound system of claim 1 wherein said transducer further comprises:
a digital signal processor (DSP) operable for additional processing on beam formed signals to and from said digital cable.

3. The ultrasound system of claim 1 wherein said transducer further comprises:
a digital signal processor (DSP) operable for additional processing on beam formed signals to and from said digital cable; and
additional backend circuitry to provide video type data to and from said digital cable.

4. The ultrasound system of claim 1 wherein said digital cable utilizes a serial interface.

5. The ultrasound system of claim 4 wherein said serial interface is implemented with LVDS technology.

6. The ultrasound system of claim 1 further comprising:
mixed-mode ASICS for controlling said signal energy.

7. The ultrasound system of claim 1 wherein said digital cable is implemented as a USB interface.

8. The ultrasound system of claim 1 wherein said digital cable is implemented as an IEEE1394 interface.

9. A method of processing ultrasonic signals, said method comprising:
placing a transducer having a plurality of elements adjacent a patient's body and injecting ultrasound from said scanheads into said patient's body, said injected ultrasound following a determined radiation pattern formed between said plurality of individual elements;
beam forming power signals to form said injected radiation pattern;
digitally processing signals to and from said beam forming;
sending digital processed signals over a digital cable having a length in excess of three feet to a processing device located separate from said transducer;
forming, according to at least one protocol and under control of a processor located in said processing device, digital images of signals received over said digital cable; and
displaying visual images of formed ones of said digital images.

10. The method of claim 9 wherein said digital cable is implemented utilizing a serial digital interface.

11. The method of claim 9 wherein said serial interface is implemented with LVDS technology.

12. The method of claim 9 wherein said digital cable is implemented as a USB type interface.

13. The method of claim 9 wherein said digital cable is implemented as a 1394 interface.

14. The method of claim 10 further comprising:
using mixed-mode ASICS between said elements and said beam forming.

15. The method of claim 10 wherein said display device is external to both said transducer and said external processing device.

16. A handheld ultrasonic device, said device comprising:
a transducer having a plurality of scanheads for placement adjacent a patient's body, said transducer operable for injecting ultrasound from said scanheads into said patient's body, said injected ultrasound injected according to a determined radiation pattern formed between said plurality of individual scanheads;
a beam former for establishing said determined radiation pattern;
a digital cable interfacing said device with an external processor; and
at least one digital signal processor interposed between said beam former and said cable.

17. The handheld ultrasonic device of claim 16 wherein said transducer is further operable for receiving back from said patient's body image signals created by said injected ultrasonic signals; and wherein said beam former and said at least one digital signal processor processes said received signals so as to reduce the bandwidth of said signals over said cable.

18. The handheld ultrasonic device of claim 17 wherein said bandwidth is reduced to at below 40 Mbps.

19. The handheld ultrasonic device of claim 16 wherein said digital signal processor comprises:
a processor for forming, according to at least one protocol, digital images of signals received over said digital cable, and
displaying visual images of said formed images.

20. The handheld ultrasonic device of claim 18 wherein said displaying is external to both said handheld device and said external processor.

21. The handheld ultrasonic device of claim 18 wherein data over said digital cable utilizes a serial interface.

22. The handheld ultrasonic device of claim 20 wherein said serial interface uses LVDS implementation.

23. The handheld ultrasonic device of claim 17 further comprises:
ASICS for controlling the establishment of said determined beam radiation pattern.

24. The handheld ultrasonic device of claim 23 wherein said ASICS are mixed-mode ASICS.

25. The handheld ultrasonic device of claim 18 wherein said digital cable is a USB cable.

26. The handheld ultrasonic device of claim 18 wherein said digital cable is implemented as a IEEE1394 interface.

27. A handheld ultrasonic device, said device comprising:
means for injecting ultrasound into a patient's body, said injected ultrasound injected according to a determined radiation pattern;
means for establishing said determined radiation pattern;
a digital cable interfacing said device with an external processor; and
means interposed between said establishing means and said cable for processing signals to and from said cable.

28. The handheld ultrasonic device of claim 27 wherein said injecting means is further operable for receiving back from said patient's body image signals created by said injected ultrasonic signals; and wherein said establishing means and said processing means reduce the bandwidth of said signals over said cable.

29. The handheld ultrasonic device of claim 28 wherein said external processor comprises:
means for forming, according to at least one protocol, digital images of signals received over said digital cable; and
means for displaying visual images of said formed images.

30. The handheld ultrasonic device of claim 29 wherein said displaying is external to both said handheld device and said external processor.

31. The handheld ultrasonic device of claim 29 wherein said digital cable is a USB cable, using USB protocols.

32. The handheld ultrasonic device of claim 28 wherein said digital cable uses a serial interface.

33. The handheld ultrasonic device of claim 28 wherein said serial interface uses LVDs implementation.

34. The handheld ultrasonic device of claim 27 wherein said radiation pattern establishing means comprises, at least in part, ASICS.

35. The handheld ultrasonic device of claim 34 wherein said ASICS are mixed-mode ASICS.
